(19) United States
(12) Patent Application Publication
(10) Pub. No.: US 2014/0228417 A1
(43) Pub. Date: Aug. 14, 2014

(54) PROCESS AND INTERMEDIATES FOR THE PREPARATION OF SUBSTITUTED 2-ARYLTHIAZOLE CARBOXYLIC ACIDS

(71) Applicants: Michael Mizhiritskii, Rehovot (IL); Ehud Marom, Kfar Saba (IL); Shai Rubnov, Tel Aviv (IL)

(72) Inventors: Michael Mizhiritskii, Rehovot (IL); Ehud Marom, Kfar Saba (IL); Shai Rubnov, Tel Aviv (IL)

(73) Assignee: MAPI PHARMA LTD., Ness Ziona (IL)

(21) Appl. No.: 14/349,983

(22) PCT Filed: Jan. 23, 2012
(86) PCT No.: PCT/IL12/50018

§ 371 Date: Apr. 4, 2014

Related U.S. Application Data
(60) Provisional application No. 61/543,341, filed on Oct. 5, 2011.

(51) Int. Cl. C07D 275/03; A61K 31/426

(52) U.S. Cl. CPC C07D 275/03; A61K 31/426
USPC 514/365; 548/201

(57) ABSTRACT

The present invention relates to processes and intermediates for the preparation of derivatives of 2-arylthiazole such as Febuxostat and its analogs. Febuxostat which is an inhibitor of xanthine oxidase, is used for the treatment of chronic hyperuricaemia in conditions in which urate deposition has occurred, such as gouty arthritis.

### FIELD OF THE INVENTION

The present invention relates to processes for the preparation of 2-arylthiazole carboxylic acid derivatives, in particular Febuxostat and its analogs.

### BACKGROUND OF THE INVENTION

Hyperuricaemia—defined as a serum uric acid concentration exceeding the limit of solubility, predisposes affected individuals to gout, a disease characterized by the formation of crystals of monosodium urate or uric acid from supersaturated fluids in joints and other tissues. Crystal deposition is asymptomatic, but is revealed by bouts of joint inflammation—gouty attacks. If left untreated, further crystals accumulate in the joints and can form deposits known as tophi. A major aim in gout management is the long-term reduction of serum uric acid concentrations below saturation levels, as this results in dissolution of crystals and their eventual disappearance. According to the guidelines of the European League Against Arthritis, the treatment goal for chronic gout is to reduce and maintain serum uric acid levels below 6 mg/dl.

Allopurinol, a xanthine oxidase inhibitor, has been the mainstay of uric-acid-lowering therapy for more than three decades. However, a significant proportion of patients receiving allopurinol do not achieve the desired reduction in serum uric acid levels, and the side effects of the drug, although uncommon, can be severe and are more frequent in patients with renal impairment.

Xanthine oxidase catalyses the last two steps of purine catabolism in humans: the oxidation of hypoxanthine to xanthine and of xanthine to uric acid. Allopurinol is an analogue of hypoxanthine. Studies of its mode of action—which involves oxidation to the species actually responsible for inhibition—suggested that more potent xanthine oxidase inhibitors from different chemical classes might provide more effective gout treatment.

Febuxostat (2-(3-cyano-4-isobutoxyphenyl)-4-methyl-5-thiazolecarboxylic acid) is a potent, selective, non-purine inhibitor of xanthine oxidase and has a more powerful uric-acid-lowering effect than allopurinol. Febuxostat has been approved for the treatment of chronic hyperuricaemia in conditions in which urate deposition has occurred, such as gouty arthritis.

EP 0513379, JP 1993500083, U.S. Pat. No. 5,614,520 and WO 92/09279 disclose a synthetic scheme for making Febuxostat. In accordance with that scheme a reaction of 4-hydroxy-3-nitrobenzaldehyde with hydroxylamine and sodium formate in refluxing formic acid gives 4-hydroxy-3-nitrobenzonitrile, which is treated with thioacetamide in hot DMF to yield the corresponding thiobenzamide. The cyclization of this thioamide with 2-chloroacetoacetic acid ethyl ester in refluxing ethanol affords 2-(4-hydroxy-3-nitrophenyl)-4-methylthiazole-5-carboxylic acid ethyl ester with 37% yield. This derivative is alkylated at the phenolic group by means of isobutyl bromide and K₂CO₃ in hot DMF, providing the 2-(4-isopropoxy-3-nitrophenyl)-4-methyl-5-thiazolecarboxylic acid in 65% yield. The reduction of the nitro group with hydrogen over Pd/C in ethanol/ethyl acetate gives the expected amino derivative, which is converted into 2-(3-cyano-4-isobutoxyphenyl)-4-methylthiazole-5-carboxylic acid ethyl ester by diazotation with NaNO₂/HCl and treatment with CuCN and KCN (the yield is 42% after silica gel chromatography). Finally, this compound is hydrolyzed with NaOH in hot THF/water, giving the end product, 2-(3-cyano-4-isopropoxyphenyl)-4-methyl-5-thiazolecarboxylic acid, in 80% yield. The above-described method for the synthesis of Febuxostat has several disadvantages including low yields (8-10% overall yield), multistage process (7 steps), and the use of problematic (thioacetamide is known as a carcinogen) or non-commercial (4-hydroxy-3-nitrobenzaldehyde) reagents.

JP 1994345724 (JP 6-345724) and a publication by Hasegawa, Heterocycles 1998, 47: 857-864 discloses a synthetic scheme for making Febuxostat. In accordance with this synthetic scheme, 2-(3-cyano-4-isoutoxy-phenyl)-4-methylthiazole-5-carboxylic acid is made by reacting 4-nitrobenzonitrile with KCN in hot DMSO, followed by a treatment with isobutyl bromide and K₂CO3, giving 4-isobutoxybenzene-1,3-dicarbonitrile, which reacts with thioacetamide in hot DMF to yield 3-cyano-4-isobutoxythiobenzamide. Cyclization of 3-cyano-4-isobutoxythiobenzamide with 2-chloroacetoacetic acid ethyl ester in refluxing ethanol affords 2-(3-cyano-4-isobutoxy-phenyl)-4-methylthiazole-5-carboxylic acid ethyl ester, which is further hydrolyzed with NaOH to the end product (35% yield) with chromatographic purifications after each step.

According to JP 10-045733, cyclization of 4-hydroxythiobenzamide with 2-bromoacetoacetic acid ethyl ester in refluxing ethanol provides 2-(4-hydroxyphenyl)-4-methylthiazole-5-carboxylic acid ethyl ester, which is formylated by a reaction with hexamethylenetetramine (HMTA) and polyphosphoric acid (PPA) in hot AcOH/water to afford 2-(3-formyl-4-hydroxyphenyl)-4-methylthiazole-5-carboxylic acid ethyl ester. The alkylation of this compound with isobutyl bromide, K₂CO₃ and KI in DMF gives 2-(3-formyl-4-isobutoxyphenyl)-4-methylthiazole-5-carboxylic acid ethyl ester, which is treated with formic acid, sodium formate and hydroxylamine hydrochloride to give the 2-(3-cyano-4-isobutoxyphenyl)-4-methyl-thiazole-5-carboxylic acid ethyl ester. Finally, this compound is hydrolyzed with NaOH in THF/EtOH.

Alternatively, 2-(3-formyl-4-hydroxyphenyl)-4-methylthiazole-5-carboxylic acid ethyl ester can also be treated first with formic acid, sodium formate and hydroxylamine hydrochloride to provide 2-(3-cyano-4-hydroxyphenyl)-4-methylthiazole-5-carboxylic acid ethyl ester, which is then alkylated with isobutyl bromide as previously described to give the 2-(3-cyano-4-isobutoxyphenyl)-4-methylthiazole-5-carboxylic acid ethyl ester [JP 10-045733].

Preparation of Febuxostat is also disclosed in JP10-139770. 4-isobutoxybenzonitrile is brominated with bromine in acetic acid (r.t., 30 h) to afford 4-isobutoxy-3-bromobenzonitrile, which is transformed to 3-bromo-4-isobutoxythiobenzamide by a reaction with thioacetamide in hot DMF. Interaction of 3-cyano-4-isobutoxythio-benzamide with 2-chloroacetoacetic acid ethyl ester in refluxing ethanol affords 2-(3-bromo-4-isobutoxy-phenyl)-4-methylthiazole-5-carboxylic acid ethyl ester, which is reacted with a compound represented by the formula MCN (M is an alkali metal or trimethylsilyl) in the presence of a catalytic amount of a nickel or a palladium complex [e.g. tetrakis(triphenyphosphine)nickel] (80° C., 16 h) to yield the 2-(3-cyano-4-isobutoxy-phenyl)-4-methylthiazole-5-carboxylic acid ethyl ester (73%), which is hydrolyzed to Febuxostat by basic catalysis.

Alternatively, it was proposed [Canivet et al., Org. Lett., 2009, 11(8): 1733-1736] to prepare Febuxostat by cross-coupling tert-butyl 4-methylthiazole-5-carboxylate with 5-iodo-2-isobutoxybenzonitrile in the presence of the Ni(OAc)₂/bipy catalyst and LiOt-Bu in sealed vessel at 100° C. for 40 h. After chromatographic purification, tert-butyl 2-(3-cyano-4-isobutoxyphenyl)-4-methylthiazole-5-carboxylate is treated with CF₃CO₂H to afford Febuxostat in 51% overall yield.

PCT patent publication no. WO 2011/073617 discloses a process for preparing Febuxostat by condensing a boronic acid derivative with a thiazole carboxylic acid alkyl or aryl ester to form an alkyl or aryl ester of Febuxostat, and hydrolyzing the ester to form Febuxostat.

It can be seen from the above, that existing methods for making Febuxostat suffer from low yields, prolonged reaction times (20 to 40 hours) and the use of problematic reagents, only some of which are commercially available, or possess hazard properties. Therefore, there continues to be a need in the art for a practical method for making Febuxostat, which not only avoids the problems of the existing art, but will be also safe, cost effective, and industrially feasible.

### SUMMARY OF THE INVENTION

The present invention relates to processes for the synthesis and isolation of 2-(3-cyano-4-isobutoxyphenyl)-4-methyl-5-thiazolecarboxylic acid (Febuxostat) represented by the structure of formula (1) and related thiazolo carboxylic acid derivatives of formula (I).

As contemplated herein, the present invention generally relates to a process for the preparation of a substituted 2-arylthiazolcarboxylic acid of formula (I):

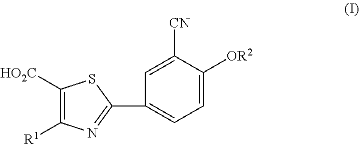

wherein

R¹ and R² are each an unsubstituted or substituted alkyl.

The method comprising the steps of:

(a) coupling a thiazole carboxylic acid derivative of formula (III):

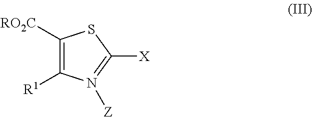

with an aryl derivative of formula (II)

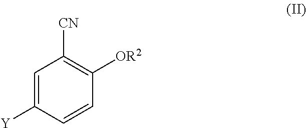

to form a 2-arylthiazolcarboxylic acid derivative of formula (IV):

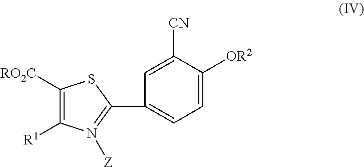

wherein R is H or Si(Ra)₃ wherein each R^{a} is independently of the other an unsubstituted or substituted alkyl, arylalkyl or aryl;

R¹ and R² are as defined above,

X, Y and Z are as described in option (i) or option (ii) hereinbelow:
either (i) X is Hal or OSO₂R′ wherein R′ is an unsubstituted or substituted alkyl, alkylaryl or aryl, Z is absent and Y is B(OR″)₂ wherein R″ is H or an unsubstituted or substituted alkyl or aryl; or
(ii) X is absent, Z together with the nitrogen to which is attached forms an N-oxide moiety of the formula ^{⊕}N—O^{⊖}; and Y is Hal or OSO₂R′ wherein R′ is as defined above;

(b) when Z together with the nitrogen to which it is attached forms an N-oxide, reducing the compound of formula (IV); and

(c) optionally, when R is Si(R^{a})₃, converting the resultant compound of step (a) or (b) to a compound of formula (I).

In one embodiment, R=H. In accordance with this embodiment, the compound of formula I (e.g., Febuxostat) is obtained directly from steps (a) or (b), and step (c) is not performed. This process offers a significant advantage over the process of WO 2011/073617, since the hydrolysis step is not needed, thus making the reaction shorter and more efficient. The finding that the coupling step (a) can be performed with the free carboxylic acid was surprising and unexpected and represents one embodiment of the present invention.

In other embodiments, R is Si(R^{a})₃. R^{a} may be an alkyl, aryl, alkylaryl, or a combination thereof. For example, R may be a trialkylsilyl, triarylsilyl, diarylalkyl silyl, dialkylarylsilyl, and the like, non-limiting examples of which are trimethylsilyl (TMS), triethylsilyl (TES), tripropylsilyl, triisopropylsilyl, triphenylsilyl, di-t-butyldimethyl silyl (TBDMS) or tert-butyldiphenylsilyl (TBDPS), with each possibility representing a separate embodiment of the present invention. In accordance with such embodiments step (c), i.e., the step of converting the silyl ester moiety CO₂Si(R^{a})₃ to the corresponding carboxylic acid CO₂H can be performed by any manner known in the art. For example, the silyl group can be removed by hydrolysis. Examples of conditions for silyl group removal include, but are not limited to acidic conditions (e.g., AcOH, BF₃, 10-CSA (camphorsulfonic acid)), or basic conditions such with fluoride ion, e.g., HF-pyridine or tetrabutylammonium fluoride (TBAF). Each possibility represents a separate embodiment of the present invention.

In one currently preferred embodiment, R¹ in Formula (I) is methyl and R² is isobutyl, and the compound of formula (I) is Febuxostat represented by the structure of formula (I):

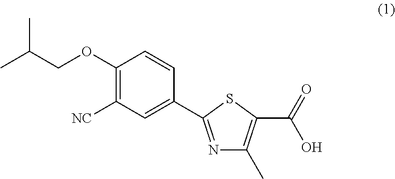 [SMILES: Cc1nc(-c2ccc(OCC(C)C)c(C#N)c2)sc1C(=O)O]

In accordance with a first alternative embodiment of the above-described process, X in compound (III) is Hal or OSO₂R′ wherein R′ is as described above, Z is absent and Y in compound (III) is B(OR″)₂ wherein R″ is as described above; In accordance with this embodiment, the present invention provides a process for preparing a substituted 2-arylthiazolcarboxylic acid of formula (I), comprising the following steps:

(a) coupling a thiazole carboxylic acid of formula (3′)

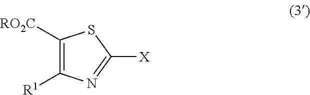

with an aryl boronic acid derivative of formula (2′)

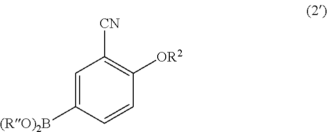

so as to generate a 2-arylthiazolcarboxylic acid derivative of formula (4′);

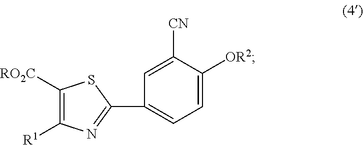

wherein R is H or Si(Ra)₃ wherein each R^{a} is independently of the other an unsubstituted or substituted alkyl, arylalkyl or aryl;

R¹ and R² are each an unsubstituted or substituted alkyl;

X is Hal or OSO₂R′ wherein R′ is an unsubstituted or substituted alkyl, alkylaryl or aryl, R″ is H or an unsubstituted or substituted alkyl or aryl; and

(b) optionally, when R is Si(R^{a})₃, converting the 2-arylthiazolcarboxylic acid silyl ester of formula (4′) to a substituted 2-arylthiazolcarboxylic acid of formula (I). The conversion in step (b) can be effectuated by removal of the silyl protecting group as described hereinabove. Alternatively, when R=H, step (b) is not performed. Each possibility represents a separate embodiment of the invention.

Preferably, the compound of formula (I) is Febuxostat of formula (1) and the process comprises the steps of:

(a) coupling a thiazole carboxylic acid of formula (3)

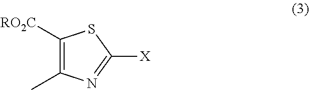

with an aryl boronic acid derivative of formula (2)

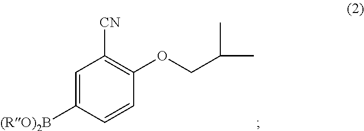

so as to generate a 2-arylthiazolcarboxylic acid derivative of formula (4);

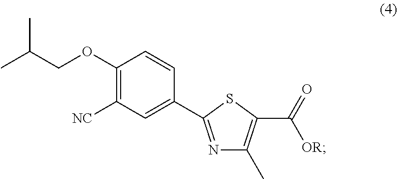

and

(b) optionally, when R is Si(R^{a})₃, converting the 2-arylthiazolcarboxylic acid silyl ester of formula (4) to Feboxostat of formula (1). The conversion in step (b) can be effectuated by removal of the silyl protecting group as described hereinabove. Alternatively, when R=H, Febuxostat is obtained directly after step (a), and step (b) is not performed. Each possibility represents a separate embodiment of the invention.

Preferably step (a) is conducted in the presence of a base and a palladium catalyst at a temperature of about room temperature (RT i.e., about 20°-25° C.) to reflux in a solvent. The base is preferably an inorganic or organic base selected from sodium carbonate, potassium carbonate, sodium hydroxide, potassium hydroxide, sodium ethoxide, potassium tert-butoxide, sodium methoxide, potassium fluoride and cesium fluoride. Each possibility represents a separate embodiment of the invention. The palladium catalyst is preferably tetrakis(triphenylphosphine)palladium, dichlorobis(triphenylphosphine)palladium, or 1,1′-bis(diphenylphosphino)ferrocene palladium chloride. Each possibility represents a separate embodiment of the invention.

Alternatively, step (a) is conducted in the presence of an ionic liquid (preferably [BMIM][BF₄]) and a palladium catalyst.

In one embodiment, the 2-X-thiazole carboxylic acid of formula (3) or (3′) is 2-bromo-4-methylthiazole-5-carboxylic acid. In another embodiment, the arylboronic acid of formula (2) or (2′) is 3-cyano-4-isobutoxyphenylboronic acid. Each possibility represents a separate embodiment of the invention.

In accordance with a second alternative embodiment of the process of the invention, X is absent, Z together with the nitrogen to which is attached forms an N-oxide moiety of the formula ^{⊕}N—O^{⊖}; and Y is Hal or OSO₂R′ wherein R′ is as defined above. In accordance with this embodiment, the present invention provides a process for preparing a substituted 2-arylthiazolcarboxylic acid of formula (I), comprising the following steps:

(a) coupling a thiazole carboxylic acid N-oxide of formula (7′):

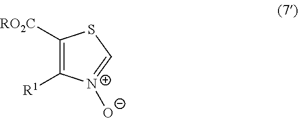

with an aryl derivative of formula (5′)

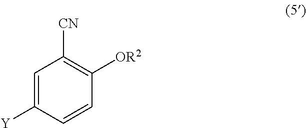

so as to generate a 2-arylthiazolcarboxylic acid derivative of formula (8′):

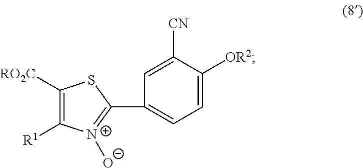

wherein R is H or Si(Ra)₃ wherein each R^{a} is independently of the other an unsubstituted or substituted alkyl, arylalkyl or aryl; and R¹ and R² are each an unsubstituted or substituted alkyl;

(b) reducing the compound of formula (8′) to a compound of formula (4′)

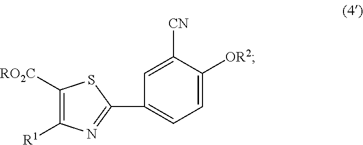

and

(c) optionally, when R is Si(Ra)₃, converting the 2-arylthiazolcarboxylic acid silyl ester of formula (4′) to a substituted 2-arylthiazolcarboxylic acid of formula (I). The conversion in step (c) can be effectuated by removal of the silyl protecting group as described hereinabove. Alternatively, when R=H, step (c) is not performed. Each possibility represents a separate embodiment of the invention.

Preferably, the compound of formula (I) is Febuxostat of formula (I) and the process in accordance with the second alternative embodiment comprises the steps of:

(a) coupling a thiazole carboxylic acid N-oxide of formula (7):

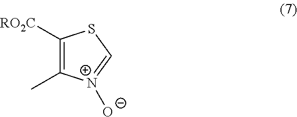

with an aryl derivative of formula (5)

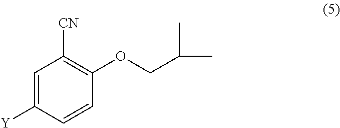

wherein R is as defined above so as to generate a 2-arylthiazolcarboxylic acid derivative of formula (8):

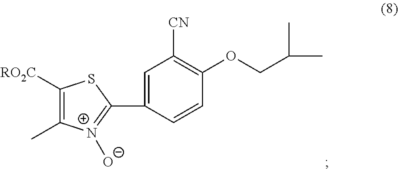

(b) reducing the compound of formula (8) to a 2-arylthiazolcarboxylic acid derivative of formula (4);

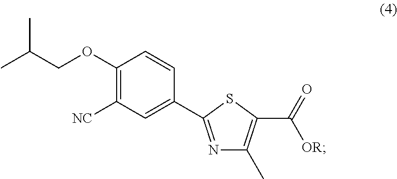

and

(c) optionally, when R is Si(R^{a})₃, converting the 2-arylthiazolcarboxylic acid silyl ester of formula (4) to Feboxostat of formula (1). The conversion in step (c) can be effectuated by removal of the silyl protecting group as described hereinabove. Alternatively, when R=H, Febuxostat is obtained directly from step (b), and step (c) is not performed. Each possibility represents a separate embodiment of the invention.

In one embodiment, the process in accordance with the second alternative embodiment further comprises the step of preparing the N-oxide derivative of formula (7′) by oxidizing a thiazole-5-carboxylic acid of formula:

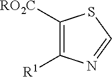

using an oxygen transfer agent. The oxygen transfer agent is preferably a hydrogen peroxide-urea complex in the presence of a carboxylic acid anhydride, and the reaction is carried out in an organic solvent at a temperature range of about 0°-60° C.

In one embodiment, step (a) in the second alternative embodiment is carried out in the presence of an organometallic catalyst and a ligand in an organic solvent with an addition of a pivalic acid salt. The organometallic catalyst is preferably palladium acetate and the ligand is preferably 2-(diphenylphosphino-2′-(N,N dimethylamino) biphenyl. Each possibility represents a separate embodiment of the invention.

In another embodiment of the second alternative process, the reduction step in step (b) is conducted in the presence of reagent selected from the group consisting of ammonium formate/Pd/C, iron dust in acetic acid, and zinc dust/ammonium chloride in water and a water miscible solvent. Each possibility represents a separate embodiment of the invention.

In some embodiments, X in compound (III) as used in the various embodiments of the process of the invention is selected from the group consisting of Cl, Br, I, OMs (O-mesylate), OTs (O-tosylate) and OTf (O-triflate). Each possibility represents a separate embodiment of the invention.

In some embodiments, Y in compound (1I) as used in various embodiments of the process of the invention is selected from the group consisting of Cl, Br, I, OMs, OTs and OTf.

In other embodiments, Y in compound (1I) as used in various embodiments of the process of the invention is B(OR″)₂ wherein R″ is preferably hydrogen (i.e., B(OR″)₂ is B(OH)₂). Each possibility represents a separate embodiment of the invention.

Each of the first and second alternative processes, as described herein, represents a separate embodiment of the present invention.

In other embodiments, the present invention relates to a compound of formula I, which is prepared by any of the processes described herein. In a preferred embodiment, the compound of formula I is Febuxostat, which is represented by the structure of formula 1.

In other embodiments, the present invention relates to a method of treating hyperuricaemia comprising administering to a subject in need thereof an effective amount of Febuxostat which is prepared by any of the processes described herein.

In other embodiments, the present invention relates to the use of Febuxostat which is prepared in accordance with any of the processes described herein, for treating hyperuricaemia.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

The present invention relates to processes for the synthesis and isolation of the 2-(3-cyano-4-isobutoxyphenyl)-4-methyl-5-thiazolecarboxylic acid (Febuxostat) and related thiazolo carboxylic acids of Formula (I). Generally, the process of the present invention can be practiced in accordance with two general alternative embodiments, referred to herein as First and Second Production methods. More specific reference to each of such alternative embodiments will now be made. It is apparent to a person of skill in the art, however, that any description provided herein is exemplary in nature and should not be construed as limiting the broad scope of the present invention.

### Chemical Definitions

An “alkyl” group refers to any saturated aliphatic hydrocarbon, including straight-chain, and branched-chain. In one embodiment, the alkyl group has 1-12 carbons designated here as C₁-C₁₂-alkyl. In another embodiment, the alkyl group has 1-6 carbons designated here as C₁-C₆-alkyl. In another embodiment, the alkyl group has 1-4 carbons designated here as C₁-C₄-alkyl. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl. Each possibility represents a separate embodiment of the present invention.

An “aryl” group refers to an aromatic ring system containing from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like. Each possibility represents a separate embodiment of the present invention.

An “alkylaryl” group is an alkyl group as defined herein bonded to an aryl group as defined herein. The aryl group can be unsubstituted or substituted through available carbon atoms with one or more groups defined hereinabove for alkyl.

A “silyl ester” group refers to a CO₂Si(R^{a})₃ group, wherein each R^{a} is independently of the other an unsubstituted or substituted alkyl, arylalkyl or aryl wherein each alkyl, arylalkyl or aryl is as defined above. Non-limiting examples of silyl esters are trimethylsilyl (TMS), triethylsilyl (TES), tripropylsilyl, triisopropylsilyl, triphenylsilyl, di-t-butyldimethyl silyl (TBDMS) or tert-butyldiphenylsilyl (TBDPS) esters.

First Production Method:

The first production method relates to a process for manufacturing compound (1) by coupling compound (2′) and compound (3′) (Scheme 1A):

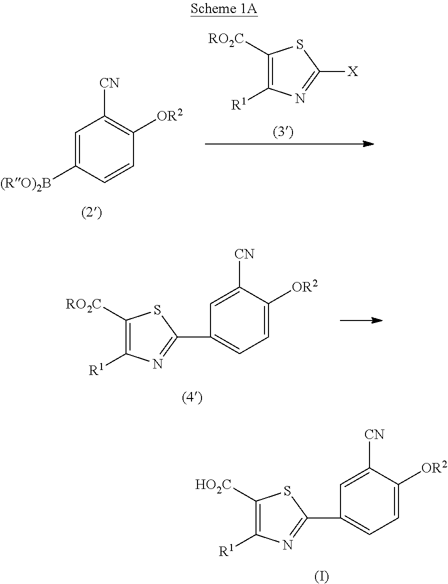

In one particular embodiment, the compound of formula (I) is Febuxostat of Formula (1), and the process comprises coupling compound (2) and compound (3) (Scheme 1B):

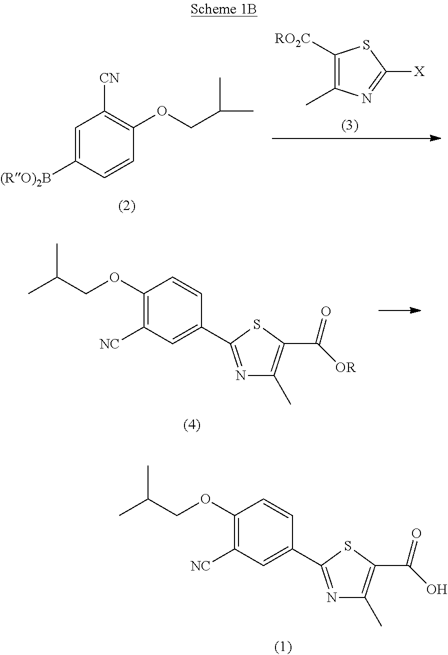

X in Schemes 1A and 1B is a Hal (i.e., a halogen) which is preferably chlorine, bromine, iodine, or the like. Alternatively, X is OSO₂R′, wherein R′ is an unsubstituted or substituted alkyl, alkylaryl or aryl, preferably X is OMs, OTs or OTf.

R″ in Schemes 1A and 1B is preferably H.

R is preferably H, in which case step 2, i.e., conversion of compound (4′) to compound (I) or compound (4) to compound (1) is not performed. R, however, may also be a silyl type protecting group (Si(R^{a})₃), wherein each R^{a} may be alkyl, aryl, or alkylaryl. For example, R may be a trialkylsilyl, triarylsilyl, diarylalkyl silyl, dialkylarylsilyl, and the like, non-limiting examples of which are trimethylsilyl (TMS), triethylsilyl (TES), tripropylsilyl, triisopropylsilyl, triphenylsilyl, di-t-butyldimethyl silyl (TBDMS) or tert-butyldiphenylsilyl (TBDPS), with each possibility representing a separate embodiment of the present invention.

For the reaction, compound (2) or (2′) and compound (3) or (3′) are preferably used in about an equimolar amount or in an excessive amount for either of the compounds and the mixture is stirred in an inert solvent under suitable reaction conditions, which can be determined by a person of skill in the art, in the presence of a base and a palladium catalyst, preferably at about room temperature (e.g., about 20° C. to 25° C.) to reflux (which temperature will depend on the nature of the solvent), generally for about 0.1 hour to about 1 day, or any period of time there between. The solvent is not particularly limited but examples thereof include aromatic hydrocarbons such as benzene, toluene, and xylene; ethers such as diethyl ether, MTBE, diisopropyl ether, tetrahydrofuran (THF), 1,4-dioxane, 1,2-dimethoxyethane (DME), and 1,2-diethoxyethane; halogenated hydrocarbons such as dichloromethane, 1,2-dichloroethane, and chloroform; alcohols such as methanol, ethanol, 2-propanol, and butanol; N,N-dimethylformamide (DMF), N-methylpyrrolidone (NMP), dimethyl sulfoxide (DMSO), water, mixed solvents thereof, and the like. Each possibility represents a separate embodiment of the present invention. As the base, inorganic bases such as sodium carbonate, potassium carbonate, sodium hydroxide, potassium hydroxide, sodium ethoxide, potassium tert-butoxide and sodium methoxide are preferred. Each possibility represents a separate embodiment of the present invention. Moreover, bases such as potassium fluoride and cesium fluoride can be used in which case it is preferable (but not required) to carry out the reaction in an aprotic solvent. As the palladium catalyst, tetrakis(triphenylphosphine)palladium, dichlorobis(triphenylphosphine) palladium, 1,1′-bis(diphenylphosphino)ferrocene palladium chloride, and the like are preferred. Each possibility represents a separate embodiment of the present invention.

Compound (2) can be prepared by any known methods, such as the methods described in EP 1783124, WO 2006/022374, WO 2006/022375, the contents of each of which are incorporated by reference herein, from 5-bromo-2-hydroxybenzonitrile or from commercially available 3-cyano-4-fluoroboronic acid. Compounds of formula (2′) can be prepared in the same or a similar manner as can be determined by a person of skill in the art.

2-Bromo-4-methylthiazole-5-carboxylic acid (3) is a commercially available compound and its preparation is described in the literature, e.g., J. Org. Chem. 2009, 74: 2578-2580; Org. Lett., 2002, 4(8): 1363; and U.S. Pat. No. 6,096,898, the contents of each of which are incorporated by reference herein. Compounds of formula (3′) can be prepared in the same or a similar manner as can be determined by a person of skill in the art.

Compounds of formula (3) or (3′) wherein R is Si(R^{a})₃ can be prepared from the corresponding carboxylic acid (compound 3 or 3′ wherein R is H) in accordance with silylation methods known to a person of skill in the art. There are many methods available for forming silyl esters. One method involves reaction of the carboxylic acid with a silyl chloride (e.g., trimethylsilyl chloride, t-butyldimethylsilyl chloride, etc.) in the presence of a base such as an amine base (e.g., trimethylamine, triethylamine, etc.), preferably at room temperature. Another method involves reaction of the carboxylic acid with a silyl triflate with a hindered base, e.g., a hindered amine base, preferably at low temperature. One reliable and rapid procedure is the Corey protocol in which the OH is reacted with a silyl chloride and imidazole at high concentration in DMF or dichloromethane. Other silylation processes for are described by C. B. Reese and E. Haslam, “Protective Groups in Organic Chemistry, “J. G. W. McOmie, Ed., Plenum Press, New York, N.Y., 1973, Chapters 3 and 4, respectively, T. W. Greene and P. G. M. Wuts, “Protective Groups in OrganicSynthesis,”2nd ed., John Wiley and Sons, New York, N.Y., 1991, Chapters 2 and 3, or Kocienski, Philip J. Protecting Groups. 3rd Ed. 2005, (2005), 679 pp., each of which is incorporated herein by reference.

Alternatively, the coupling of compound (2) and compound (3) can be performed in an ionic liquid, for example, [BMIM][BF₄], using, e.g., Pd(PPh₃)₄ as catalyst. One embodiment of this process involves pre-heating the aryl halide (3) to about 110° C. in the ionic liquid with the Pd-complex. The arylboronic acid (2) and Na₂CO₃ (2 equiv.) are later added to start the reaction. This method has the following advantages: the reaction is completed in 0.5 h with high yield of compound (1); the formation of the homo-coupling aryl by-product is suppressed; the ionic catalyst layer can be reused after the extraction of the products with methylene chloride, MTBE or ethyl acetate or like solvents, and the removal of the by-products (NaHCO₃ and NaXB(OH)₂) with excess of water. No deactivation was observed with this procedure over five further reaction cycles.

This method is more economically advantageous on an industrial scale than the methods described in patents/patent applications EP 0513379, JP 1993500083, U.S. Pat. No. 5,614,520, WO 92/09279, JP 1994345724, JP 64329647, JP 10045733, and JP 10139770, due to the fewer number of stages and the simplicity of execution.

When R is H, Febuxostat is obtained directly from step (a), and there is no need to form the carboxylic acid in a separate step. However, in another embodiment, R is Si(R^{a})₃. In accordance with such embodiment the step of converting the silyl ester moiety CO₂Si(R^{a})₃ to the corresponding carboxylic acid CO₂H can be performed by any manner known in the art. For example, the R group can be removed by hydrolysis. Examples of conditions for silyl group removal include, but are not limited to acidic conditions (e.g., AcOH, BF₃, 10-CSA (camphorsulfonic acid)), or basic conditions such with fluoride ion, e.g., HF-pyridine or tetrabutylammonium fluoride (TBAF).

Conversion of the silyl ester moiety to the corresponding carboxylic acid can also be performed by methods well known in the art, as described, e.g., in Greene's Protective Groups in Organic Synthesis, 4th Edition, Willey, 2006, p. 543. The contents of each of these references are incorporated by reference herein.

Second Production Method:

Canivet et al. [Org. Lett., 2009, 11(8): 1733-1736] disclose a method for preparation of Febuxostat by cross-coupling tert-butyl 4-methylthiazole-5-carboxylate with 5-iodo-2-isobutoxybenzonitrile in the presence of a Ni(OAc)₂/bipy catalyst and LiOt-Bu as a base in a sealed vessel at 100° C. for 40 h. Following chromatographic purification and, if desired, silyl ester deprotection, Febuxostat is recovered in 51% overall yield.

The industrial applicability of this reaction is poor, since the method suffers from low yields, prolonged reaction times (20 to 40 hours), harsh conditions (high temperature, problematic reagents) and requires the use of special equipment (sealed vessels), which are not commercially available or are expensive.

The applicants have unexpectedly found that the use of N-oxide (7) instead of thiazole (6) imparts a dramatic increase in reactivity in a direct arylation process. This permits high yielding, regioselective, and room temperature arylation at the C2 position of the thiazole ring (Schemes 2A and 2B):

Scheme 2A describes a process for preparing a compound of formula (I), in accordance with this second production method, by coupling a compound of formula (7′) with a compound of formula (5′) to produce a compound of formula (8′), reducing compound (8′) to a compound of formula (4′), and, if needed, hydrolyzing or cleaving the R group to generate a compound of formula (I).

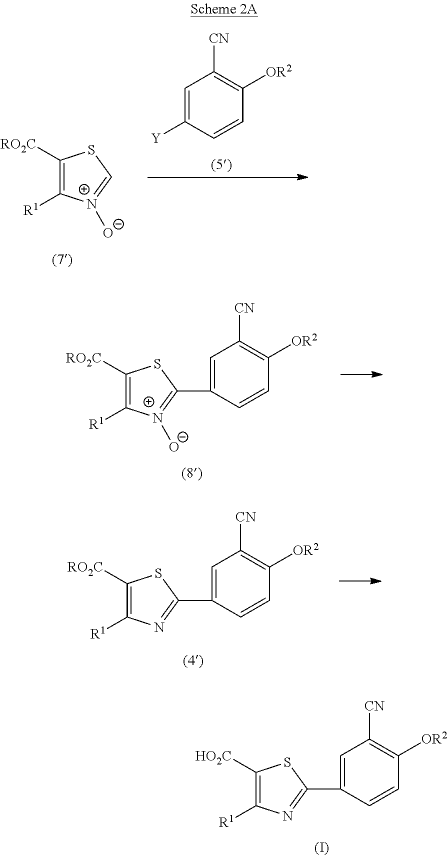

Scheme 2B describes a process for preparing a compound of Febuxostat of formula (1) in accordance with this second production method.

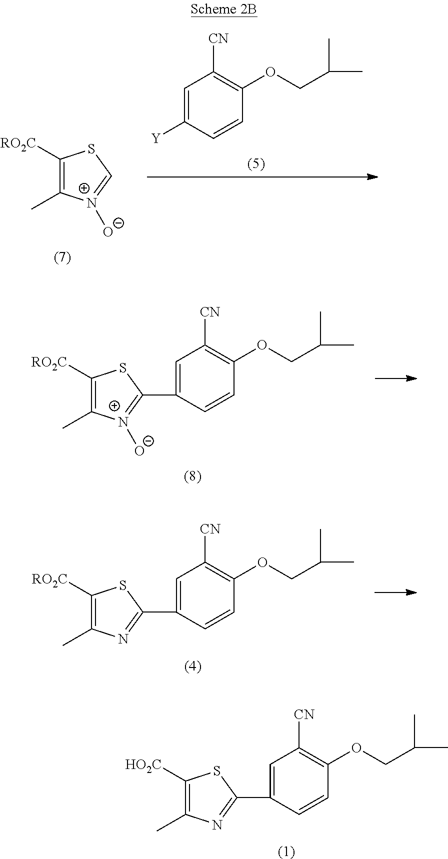

Scheme 2C shows an embodiment of the process of the invention in comparison with the method described in the literature, which involves coupling a compound of formula (6) with a compound of formula (5):

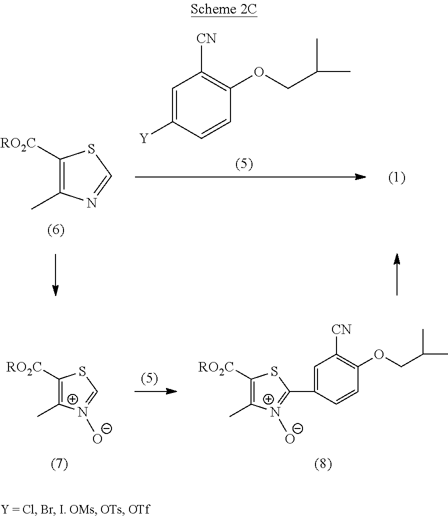

The process of the invention is unexpectedly advantageous over the process described by Canivet et al., since the high overall yield (86%), mild reaction conditions and available reagents make this approach industrially useful not only for Febuxostat production, but also for the preparation of other arylthiazole biologically active compounds such as Sodelglitazar and Amythamicin D, among others.

The present production method is a method for producing compound (1) of the invention by:

a) Coupling of compound (7) and compound (5) or compound (7′) and compound (5′) to form N-oxide (8) or (8′), respectively; and

b) Reduction of compound (8) or (8′) to a compound of formula (4) or (4′), respectively.

When R=H, the process directly results in compound (1) (Febuxostat). When R is a silyl group, however, the process further comprises step c:

c) Conversion of the silyl ester in compound (4) or (4′) to the corresponding carboxylic acid by removal of the silyl ester moiety by, e.g., hydrolysis to afford the compound of formula (1) or (I) (Schemes 2A and 2B). Removal of the silyl protecting group can be effectuated by any one of the methods described above for the First Production Method.

The compound of formula (7) or (7′) can be prepared by oxidation of thiazole (6) or (6′), respectively.

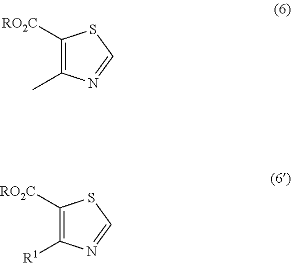

wherein R¹ is an unsubstituted or substituted alkyl.

Oxidation Step:

Transformation of thiazole (6) or (6′) to the corresponding N-oxide (7) or (7′) can be performed by reacting thiazole (6) or (6′) with an oxygen transfer agent, such as inorganic and organic peracids (e.g., meta-chloroperbenzoic acid (mCPBA), permaleic acid and the like), hydrogen peroxide in the presence of catalysts, such as MeReO₃ [as described in J. Org. Chem. 1998, 63: 1740], oxone, dimethyldioxirane, hypohalogenides, such as complex of hypofluoride and acetonitrile (HOF.CH₃CN) [as described in Chem. Commun., 2006, 2262-2264], using an oxygen transfer agent in an equimolar amount or in excess. The contents of the aforementioned references are incorporated by reference herein. Each possibility represents a separate embodiment of the present invention.

Preferably, the oxygen transfer agent is a hydrogen peroxide-urea complex in the presence of a carboxylic acid anhydride, preferably, in the presence of trifluoroacetic anhydride. The reaction is carried out in a suitable solvent at an exemplary temperature range of about 0°-60° C., preferably, at 20°-25° C. The solvent is not particularly limited but examples thereof include aromatic hydrocarbons such as benzene, toluene, and xylene; ethers such as diethyl ether, MTBE, diisopropyl ether, tetrahydrofuran (THF), 1,4-dioxane, 1,2-dimethoxyethane, and 1,2-diethoxyethane; halogenated hydrocarbons such as dichloromethane, 1,2-dichloroethane, and chloroform; alcohols such as methanol, ethanol, 2-propanol, and butanol, hexafluoro-2-propanol, N,N-dimethylformamide (DMF), N-methylpyrrolidone (NMP), dimethyl sulfoxide (DMSO), mixed solvents thereof, and the like, preferably, halogenated hydrocarbons and hexafluoro-2-propanol, most preferably, dichloromethane. Each possibility represents a separate embodiment of the present invention.

Step (a)—(Coupling)

The applicants have surprisingly found that the coupling of compound (7) or (7′) and compound (5) or (5′) to form N-oxide (8) or (8′), respectively, can be performed with high yield of compound (8) or (8′) (>90%) if it is carried out in the presence of an organometallic catalyst, preferably, palladium acetate in the presence of a ligand, preferably, 2-(diphenylphosphino-2′-(N,N dimethylamino) biphenyl in an organic solvent, preferably, toluene, by addition of pivalic acid salts, such as potassium pivalate, sodium pivalate or cesium pivalate, preferably, cesium pivalate; most preferably cesium pivalate generated in situ by reacting pivalic acid (PivOH) with excess of cesium carbonate. The reaction temperature is typically about 20°-30° C.

The product of the reaction can separated from the reactants by a water-solvent extraction, preferably, from an acidic aqueous solution by extraction with toluene. The Palladium and the ligand, 2-(diphenylphosphino-2′-(N,N dimethylamino) biphenyl, can be separated from the aqueous solution and recycled.

It is apparent to a person of skill in the art that the aforementioned method is representative and does not limit the broad scope of the present invention. Other coupling methods can be used to produce the compounds of formula (8) or (8′), for example, the methods of CA 2569943 and US 2008/132698, which disclose the preparation of biaryl compounds by coupling aryl N-oxides with Ar—X compounds in the presence of palladium catalysts, a phosphorous donor ligand or a N-heterocyclic carbene ligand and copper salts. The coupling reaction proceeds in the presence of a base such as K₂CO₃, NaOH, KOH and K₃PO₄ in an organic solvent such as aromatic solvent, dioxane, mesitylene, N,N-dimethylacetamide, N,N-dimethylformamide, N-methylpyrrolidinone, tetrahydrofuran, dichloromethane, ether or a mixture thereof at a temperature of about 80° C. to 130° C. The contents of the aforementioned references are incorporated by reference in their entirety.

Step (b)—(Reduction):

The Reduction of N-oxide (8) to compound (1) can be achieved by any of the methods known in the art, for example, by ammonium formate/Pd/C [US2008132698; J. Am. Chem. Soc., 2009, 131(9): 3291], iron dust in acetic acid, zinc dust in THF/ammonium chloride aqueous solution [Am. Chem. Soc., 2009, 131(9): 3291]. The contents of the aforementioned references are incorporated by reference herein.

Optional Step (c)—Conversion of Silyl Ester to Acid:

Conversion of the silyl ester moiety CO₂Si(R^{a})₃ to the corresponding carboxylic acid CO₂H can be effectuated by any manner known in the art as described in above for the First Production Method.

The principles of the present invention are demonstrated by means of the following non-limitative examples.

### EXAMPLES

Specific compounds which are representative of this invention were prepared as per the following examples and reaction sequences; the examples and the figures depicting the reaction sequences are offered by way of illustration, to aid in the understanding of the invention and should not be construed to limit in any way the invention set forth in the claims which follow thereafter.

No attempt has been made to optimize the yields obtained in any of the reactions. Unless otherwise noted, the materials used in the examples were obtained from readily available commercial suppliers or synthesized by standard methods known to one skilled in the art of chemical synthesis.

### Example 1

### Preparation of Febuxostat from 2-(4-isobutoxy-3-isocyanophenyl)-4-methylthiazole-5-carboxylic acid and (3-cyano-4-isobutoxyphenyl)boronic acid

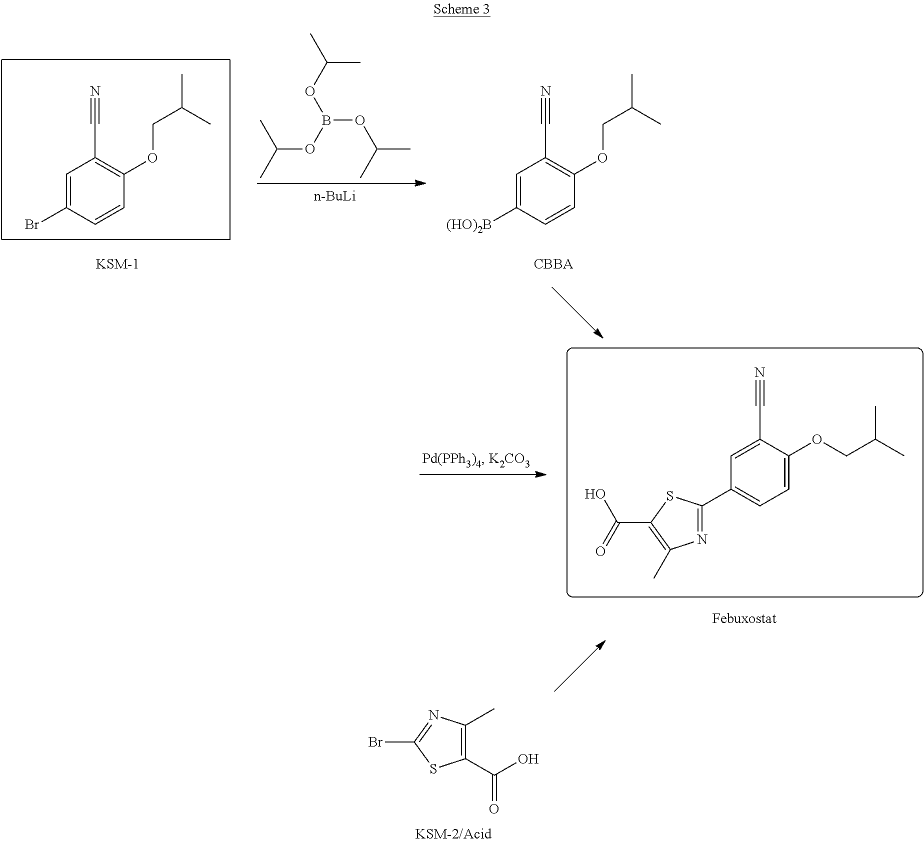

(3-cyano-4-isobutoxyphenyl)boronic acid (CBBA) was prepared from 5-bromo-2-hydroxybenzonitrile in accordance with known methods as described above. Briefly, 100 g of KSM-1 (1.0 eq.), 118 g of triisopropyl borate (1.6 eq.), and 600 ml dry (THF) were charged in a round bottom flask and the reaction was cooled to −65° C. 397 ml n-BuLi (15% in n-nexane) were added dropwise slowly at −65° C. over a period of 60 minutes, and the reaction was maintained at −65° C. for 3 to 4 hours. After completion of the reaction, the temperature was raised to −20° C. and the reaction mixture was quenched with 2N HCl (the temperature rose up to 0°-5° C. during quenching). The organic layer was separated by extraction with ethyl acetate, concentrated and washed with water. The crude product was taken to the next step without further purification.

In a mixed solution of 1,170 ml of dimethoxy ethane (DME), 156 ml MeOH and 122.8 g of K₂CO₃ (2.5 eq.) were dissolved 78 g of (3-cyano-4-isobutoxyphenyl)boronic acid (CBBA) obtained from step 1 (1 eq.) and 79 g of 2-bromo-4-methylthiazole-5-carboxylic acid (KSM-2/Acid, 1 eq.), and the resulting solution was degassed using N₂ for 15 min and then heated at 70°-80° C. in the presence of 16.5 g of tetrakis(triphenylphosphine) palladium (0.04 eq.) and 1.12 g tetra n-butyl ammonium bromide (TBAB, 0.02 eq.) for 12 hours under TLC or HPLC monitoring. After consumption of the starting material, the reaction mixture was cooled to 25°-30° C., the solid was filtered and washed with 160 ml DME. The wet solid was dissolved in water (780 ml), and the aqueous layer was washed with ethyl acetate (2×500 ml). Charcoal was added and the reaction was stirred for 30 minutes at 25°-30° C., then filtered and washed with water. Then the pH was adjusted to 4.6-4.8 with dilute HCl (18%). The reaction was stirred for 1 h, the solid was filtered, and washed with water. The product was stirred in water for 30 minutes at 65°-70° C., and the reaction was cooled to 25°-30° C. and stirred for 30 minutes. The solid was filtered and washed with water, then dried at 45-50° C.

The crude product (65 g) was dissolved in 450 ml methanol at 25-28° C., and the reaction was heated to 78-80° C. until a clear solution was obtained. To this, 3 g of charcoal were added, the reaction was stirred for 20-30 min at 80° C., then filtered. The bed was washed with methanol, charged into a round bottom flask, stirred for 60-90 min at 25°-30° C., then the solid was filtered and washed with methanol. The compound was dried in vacuum overnight at 50° C. The above recrystallization steps were repeated.

Alternatively, the crude product (45 g) was dissolved in DMSO at 25-28° C., and the reaction was heated to 58°-60° C. until a clear solution was obtained. To this, charcoal (5% w/w) was added, the reaction was stirred for 20 min at 60° C., then filtered. The bed was washed with DMSO, and the reaction mass was cooled to 50°-55° C. Water (45 ml) was added dropwise, and the reaction was slowly cooled to 20° C. and maintained at this temperature for 1.5 h, then cooled to 5° C. and maintained at this temperature for 20 minutes. The solid was filtered and washed with DMSO and water, then the compound was dried in vacuum overnight at 50° C.

The product was at least 99% pure by HPLC, and was confirmed by MS.

### Example 2

### Preparation of Febuxostat from 2-(4-isobutoxy-3-isocyanophenyl)-4-methylthiazole-5-carboxylic acid TBDMS ester and (3-cyano-4-isobutoxyphenyl)boronic acid

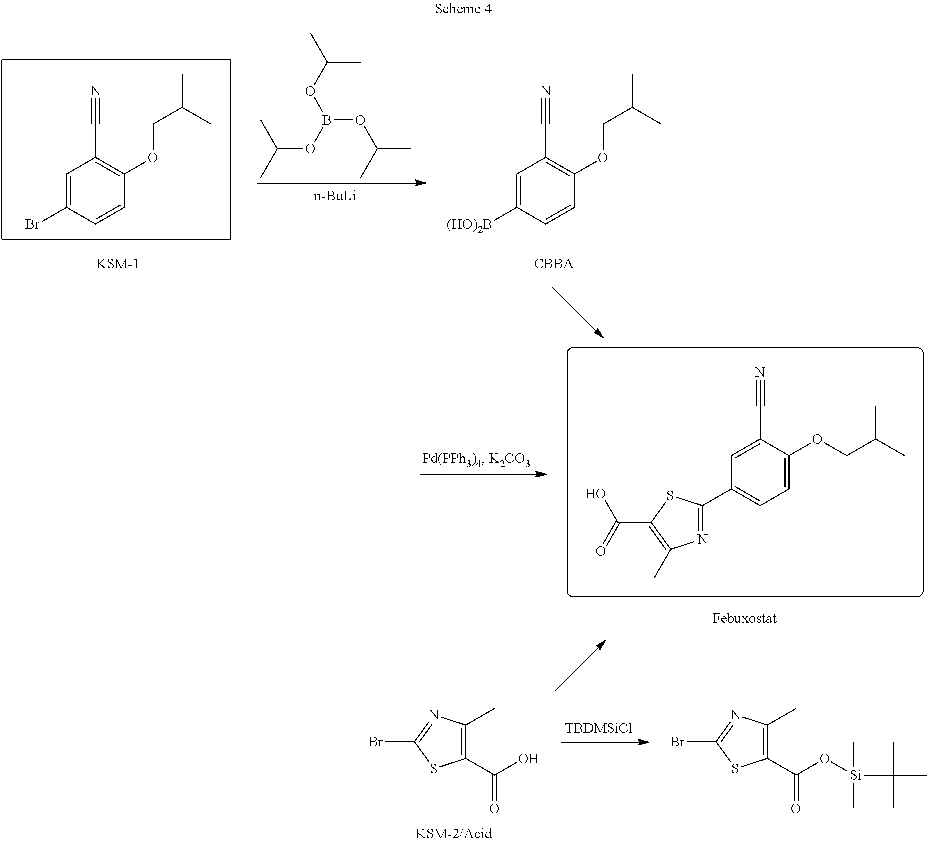

(3-cyano-4-isobutoxyphenyl)boronic acid was prepared from 5-bromo-2-hydroxybenzonitrile as described above.

To a mixture of 30 ml acetone and 3.44 g (1.5 eq.) triethylamine (TEA) were added 5 g of 2-bromo-4-methylthiazole-5-carboxylic acid (KSM-2/Acid). t-butyldimethylsilyl chloride (TBDMSiCl, 3.3 g, 1.0 eq.) was dissolved in 2 volumes of Acetone and added slowly dropwise. The reaction was heated to 50° C. and maintained for 3 hours. The product (2-bromo-4-methylthiazole-5-carboxylic acid TBMDS ester) was isolated. A similar reaction with trimethylsilyl chloride (TMS-Cl) was also performed.

Febuxostat was prepared from reaction of 2-bromo-4-methylthiazole-5-carboxylic acid TBMDS ester with (3-cyano-4-isobutoxyphenyl)boronic acid (CBBA) in accordance with the method described in Example 1.

It is appreciated by persons skilled in the art that the present invention is not limited by what has been particularly shown and described hereinabove. Rather the scope of the present invention includes both combinations and sub-combinations of various features described hereinabove as well as variations and modifications. Therefore, the invention is not to be constructed as restricted to the particularly described embodiments, and the scope and concept of the invention will be more readily understood by references to the claims, which follow.

What is claimed is:

1-23. (canceled)

24. A process for the preparation of Febuxostat represented by the structure of formula (1): 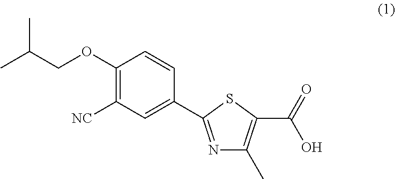 [SMILES: Cc1nc(-c2ccc(OCC(C)C)c(C#N)c2)sc1C(=O)O]
the process comprising the steps of:
(a) coupling a thiazole carboxylic acid derivative of formula (III): 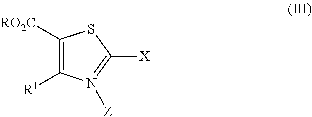
with an aryl derivative of formula (II) 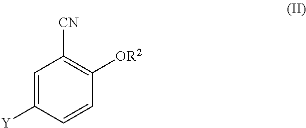
to form a 2-arylthiazolcarboxylic acid derivative of formula (IV): 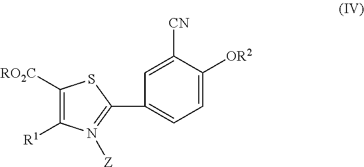
wherein
R¹ is methyl and R² is isobutyl;
R is H or Si(R^{a})₃ wherein each R^{a} is independently of the other an unsubstituted or substituted alkyl, arylalkyl or aryl;
X, Y and Z are as described in (i) or (ii) hereinbelow:
(i) X is Hal or OSO₂R′ wherein R′ is an unsubstituted or substituted alkyl, alkylaryl or aryl, Z is absent and Y is B(OR″)₂ wherein R″ is H or an unsubstituted or substituted alkyl or aryl; or
(ii) X is absent, Z together with the nitrogen to which is attached forms an N-oxide moiety of the formula ^{⊕}N—O^{⊖}; and Y is Hal or OSO₂R′ wherein R′ is as defined above;
(b) when Z together with the nitrogen to which is attached forms an N-oxide, reducing the compound of formula (IV); and
(c) optionally, when R is Si(R^{a})₃, converting the resultant compound of step (a) or (b) to Febuxostat of formula (1).

25. The process according to claim 24, wherein X is a Hal or OSO₂R′, Z is absent and Y is B(OR″)₂ wherein R′ and R″ are as defined above, and the process comprises the following steps:
(a) coupling a thiazole carboxylic acid derivative of formula (3) 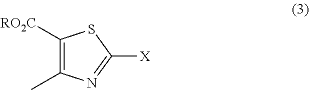
with an aryl boronic acid derivative of formula (2) 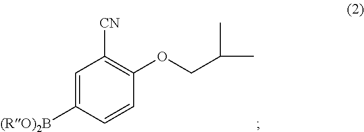
so as to generate a 2-arylthiazolcarboxylic acid derivative of formula (4); 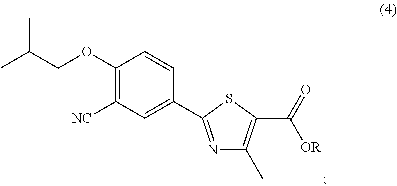
and
(b) optionally, when R is Si(R^{a})₃, converting the 2-arylthiazolcarboxylic acid silyl ester of formula (4) to Febuxostat of formula (1).

26. The process according to claim 25, wherein step (a) is conducted in the presence of a base and a palladium catalyst at a temperature of about RT to reflux in a solvent.

27. The process according to claim 26, wherein the base is an inorganic or organic base selected from sodium carbonate, potassium carbonate, sodium hydroxide, potassium hydroxide, sodium ethoxide, potassium tert-butoxide, sodium methoxide, potassium fluoride and cesium fluoride.

28. The process according to claim 25, wherein step (a) is conducted in the presence of an ionic liquid and a palladium catalyst.

29. The process according to claim 28, wherein the ionic liquid is [BMIM][BF₄]).

30. The process according to claim 26, wherein the palladium catalyst is tetrakis(triphenylphosphine)palladium, dichlorobis(triphenylphosphine)palladium, or 1,1′-bis(diphenylphosphino)ferrocene palladium chloride.

31. The process according to claim 24, wherein R is H.

32. The process according to claim 24, wherein R is Si(R^{a})₃.

33. The process according to claim 24, wherein R is selected from the group consisting of trialkylsilyl, triarylsilyl, dialkylaryl silyl, or diarylalkyl silyl.

34. The process according to claim 33, wherein R is selected from the group consisting of trimethylsilyl (TMS), triethylsilyl (TES), tripropylsilyl, triisopropylsilyl, triphenylsilyl, di-t-butyldimethyl silyl (TBDMS) and tert-butyldiphenylsilyl (TBDPS).

35. The process according to claim 24, wherein in step (a) the arylboronic acid of formula (II) or (2) is 3-cyano-4-isobutoxyphenylboronic acid.

36. The process according to claim 24, wherein in step (a) the 2-X-thiazole carboxylic acid of formula (III) or (3) is 2-bromo-4-methylthiazole-5-carboxylic acid.

37. The process according to claim 24, wherein X is absent, Z together with the nitrogen to which is attached form an N-oxide moiety of the formula: ^{⊕}N—O^{⊖}; and Y is Hal or OSO₂R′ wherein R′ is as defined above, and the process comprises the following steps:
(a) coupling a thiazole carboxylic acid N-oxide of formula (7): 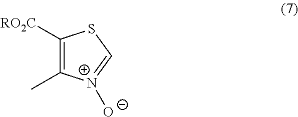
with an aryl derivative of formula (5) 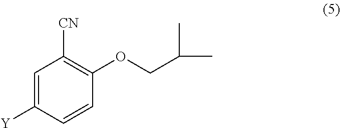
wherein R is as defined above so as to generate a 2-arylthiazolcarboxylic acid derivative of formula (8): 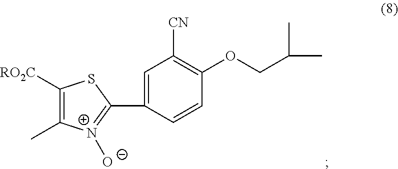
(b) reducing the compound of formula (8) to a 2-arylthiazolcarboxylic acid derivative of formula (4); 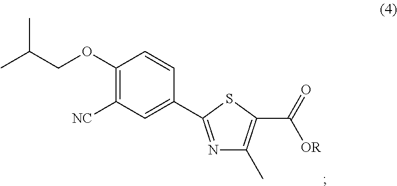
and
(c) optionally, when R is Si(R^{a})₃, converting the 2-arylthiazolcarboxylic acid silyl ester of formula (4) to Febuxostat of formula (1).

38. The process according to claim 37, wherein the N-oxide derivative of formula (7) is prepared by oxidizing a thiazole-5-carboxylic acid derivative of formula: 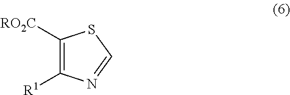
using an oxygen transfer agent.

39. The process according to claim 38, wherein the oxygen transfer agent is a hydrogen peroxide-urea complex in the presence of a carboxylic acid anhydride, and the reaction is carried out in an organic solvent at a temperature range of about 0°-60° C.

40. The process according to claim 37, wherein in step (a) the reaction is carried out in the presence of an organometallic catalyst and a ligand in an organic solvent in the presence of a pivalic acid salt.

41. The process according to claim 40, wherein the organometallic catalyst is palladium acetate and the ligand is 2-(diphenylphosphino-2′-(N,N dimethylamino) biphenyl.

42. The process according to claim 37, wherein in step (b) the reduction reagent is selected from the group consisting of ammonium formate/Pd/C, iron dust in acetic acid, and zinc dust/ammonium chloride in water and a water miscible solvent.

43. The process according to claim 24, wherein R is Si(R^{a})₃, and the conversion step comprises hydrolyzing the silyl ester group CO₂Si(R^{a})₃ to a carboxylic acid CO₂H.

44. The process according to claim 24,
wherein X in compound (III) is selected from the group consisting of Cl, Br, I, OMs, OTs and OTf; or
wherein Y in compound (II) is selected from the group consisting of Cl, Br, I, OMs, OTs and OTf; or
wherein Y in compound (II) is B(OR″)₂, wherein R″ is H.

45. Febuxostat of formula I, which is prepared by the process according to claim 24.

46. A method of treating hyperuricaemia comprising administering to a subject in need thereof an effective amount of Febuxostat which is prepared in accordance with the process of claim 24.
